(19) United States
(12) Patent Application Publication
(10) Pub. No.: US 2014/0271616 A1
(43) Pub. Date: Sep. 18, 2014

(54) Compositions And Methods For Mesenchymal And/Or Chondrogenic Differentiation Of Stem Cells

(71) Applicant: The Board of Trustees of the Leland Stanford Junior University, Palo Alto, CA (US)

(72) Inventors: Hossein Nejadnik, Atherton, CA (US); Olga Lenkov, Stanford, CA (US); Heike Daldrup-Link, San Francisco, CA (US)

(21) Appl. No.: 14/210,752

(22) Filed: Mar. 14, 2014

Related U.S. Application Data
(60) Provisional application No. 61/787,729, filed on Mar. 15, 2013.

(51) Int. Cl. C12N 5/0775; A61K 35/28; A61K 35/32

(52) U.S. Cl. CPC C12N 5/0662; A61K 35/32; A61K 35/28
USPC 424/130.1; 424/93.7; 424/184.1; 435/377; 435/366

(57) ABSTRACT

Chondrocytes and compositions including chondrocytes produced via methods that do not require the formation of embryoid bodies are disclosed herein. The cells and compositions disclosed herein are suitable for use in treating osteoarthritis and other cartilage disorders or injury, as well as for tissue regeneration, particularly cartilage regeneration.

### CROSS REFERENCE TO RELATED APPLICATIONS

This application claims the benefit of and priority to U.S. Provisional application Ser. No. 61/787,729, filed on Mar. 15, 2013, having the title “Compositions an Methods for Mesenchymal and/or Chondrogenic Differentiation of Stem Cells,” which is herein incorporated by reference as if expressed in its entirety.

### STATEMENT REGARDING FEDERALLY SPONSORED RESEARCH OR DEVELOPMENT

This invention was made with Government support under Contract Nos. R21AR059861-01 and 2R01AR054458-05 awarded by the National Institutes of Health. The U.S. Government has certain rights in this invention.

### BACKGROUND

Osteoarthritis (OA) is a major cause of disability that affects about 43 million individuals in the United States. The annual costs to society in medical care and lost wages associated with OA are in the order of $95 billion. OA-related injuries are the leading cause of disability among the elderly and are expected to increase by about 66 to about 100% by 2020. Articular cartilage defects are the major source of pain and functional impairment in patients with OA. Permanent articular cartilage defects are characterized by deterioration of the collagen matrix and depletion of aggrecan and type 2 collagen. Permanent articular cartilage defects represent the primary cause of OA and are difficult to treat because articular cartilage lacks self-regeneration capacity. Therefore, restoration of cartilage lesions presents a major challenge in OA treatment. Stem cell transplants or implants provide a therapeutic or preventative option.

Human induced pluripotent stem cells (hiPS cells or hiPSC) represent a novel cell type for cartilage regeneration based on non-pluripotent cells, such as adult fibroblasts, which are reprogrammed via introduction of transcription factors that are linked to pluripotency. hiPS cells are autologous and, thus, able to transform into patient-specific stem cells. This feature helps to avoid immune reactions and overcomes ethical concerns that are associated with human embryonic stem cell transplants. Moreover, hiPS cells also overcome limitations associated with bone marrow derived stem cells, such as invasive harvesting procedures, variable yields, and limited cartilage regeneration potential in cells obtained from older patients. hiPS cells can be harvested as a homogeneous cell population in a consistent and reproducible manner, are easily expanded, and may be better directed to form functional 3D tissue in an in vivo environment. Thus, hiPS cells currently represent the most promising cell type for cartilage restoration.

However, the full potential for the use of hiPS cells in cartilage restoration has yet to be fully realized. Current approaches for chondrogenic differentiation of hiPS cells are complicated, laborious, and inefficient, requiring generation of embryoid bodies and related differentiation into mesenchymal, endodermal, and ectodermal cell lineages with subsequent selection of a small subset of generated cells towards chondrogenic differentiation pathways. Current hiPS cell cartilage restoration methods are inefficient due in part to the fact that it is difficult to control the number and size of embryoid bodies, which leads to heterogeneous, irregular, and unpredictable differentiation of pluripotent cells to undesired cell lineages. Accordingly, improved compositions and methods for more efficient chondrogenic differentiation are needed to realize the full potential of hiPS cells.

### BRIEF DESCRIPTION OF THE DRAWINGS

Further aspects of the present disclosure will be readily appreciated upon review of the detailed description of its various embodiments, described below, when taken in conjunction with the accompanying drawings.

FIG. 1 shows the current method used for classical chondrogenic differentiation of hiPSCs via formation of embryoid bodies, outgrowth of endodermal (green), ectodermal (yellow) and mesodermal (red) cell lineages, selection of mesodermal cells, induction of MSC and induction of chondrocytes.

FIG. 2 shows one embodiment of an embryoid body-free method of generating chondrocytes from induced pluripotent stem cells (iPSCs).

FIGS. 3A-3H show immunofluorescence stainings of hiPSC with DAPI counter stains (3C, 3D, and 3F), demonstrating positive pluripotency markers NANOG (3A), OCT4 (3B), SOX2 (3E), and TRA-1-60 (3F). Bright field image (3H) confirms colony formation of the hiPSC.

FIGS. 4A-4C show representative hematoxylin and eosin (H&E) stains of a hiPSC-derived teratoma, which confirms differentiation into all three germ layers, including ectoderm (4A), mesoderm (4B), and endoderm (4C). Scale bar is equal to 200 microns.

FIG. 5 shows one embodiment of a method of culturing hiPSC on Matrigel™ coated plates and shows the colony-like growing hiPSC. On day 1, changing the mTeSR1 media to hMSC media leads to initiation of mesodermal morphology of the hiPSC and out-growing of the cells from the colonies. On about day 5, the pre-differentiated hiPSCs are sub-cultured to uncoated and untreated culture flask, demonstrating the ability of the cells to attach to the polystyrene flask. On about day 21, passage 4 of hiPSC-derived MSC-like cells demonstrates spindle shape morphology of the differentiated cells, similar to hMSC. Scale bar is equal to 200 microns.

FIGS. 6A-6L show a flow cytometry analysis of surface markers of hiPSC (6B, 6E, 6H, and 6K) and hiPSC-derived MSC-like cells (6C, 6F, 61, and 6L) at passage 4. Cells show positive hMSC surface markers according to the International Society for Cell Therapy (ISCT), including CD105 (6J-6L), CD73 (6D-6F), and CD90 (6G-6I), and lack of CD45, CD34, CD14 or CD11b, CD19, and HLA-DR (6A-6C) surface molecules. Data was normalized to unstained 6A, 6D, 6G, and 6J.

FIG. 7 shows relative gene expression of hiPSC, hiPSC-derived MSC-like cells, and chondrogenic cell pellets at about day 28 (about day 7 of chondrogenic differentiation) and about 35 (about day 14 of chondrogenic differentiation), as determined by qPCR. Data are displayed as means and standard errors of triplicate experiments per sample. Cells at about day 14 of chondrogenic differentiation show significantly increased gene expression of the chondrogenic markers COL2A1, SOX9, and aggrecan (ACAN) compared to hiPSC and hiPSC-derived MSC-like cells (*** indicates p<0.001).

FIGS. 8A-8F show histological evaluation of hiPSC-derived MSC-like cells after 21 days of chondrogenic differentiation. H&E stain (8A and 8B) shows the chondrocyte differentiation morphology. Alcian blue stain (8C and 8D) demonstrates positive glycosaminoglycan production. Immunohistochemistry shows positive stains for collagen type II (8E and 8F). Scale bar is equal to 100 μm in low magnification (8A, 8C, and 8E) and 50 μm in high magnification (8B, 8D, and 8F).

FIGS. 9A-9I show representative images from MRI evaluation of hiPSC-derived MSC-like cells (9B, 9E, and 9H), chondrogenic cell pellets (9C, 9F, and 9I), and scaffold only (as control) (9A, 9D, and 9G) at different time points (about Day 1 (9A-9C), about day 21 (9D-9F), and about day 42 (9G-9I) after implantation into osteochondral defects of the distal femur of rat knee joints. Sagittal T2-weighted MR images of the knee joint with superimposed T2 relaxation time maps of cell implants in osteochondral defects show the T2 values distribution of transplanted cells. Transplants show lower T2 values on about 21 and on about 42 days after the implantation as compared to about day 1.

FIG. 10 shows T2-relaxation times corresponding to the MRI images of FIGS. 9A-9I. Transplants show a significant decrease of T2 values at about 21 and about 42 days after implantation as compared to about day 1. This difference is higher in hiPSC-derived MSC-like cells and hiPSC-derived chondrogenic cell transplants as compared to the scaffold only. Data are displayed as means and SE of triplicate experiments. * and ** indicates p<0.05 and p<0.01 respectively.

FIGS. 11A-11I show representative images from histological evaluation of the implanted hiPSC-derived MSC-like cells (11B, 11E, and 11H) and chondrogenic pellets (11C, 11F, and 11I). H&E stain (11A-11C) of chondrogenic differentiated hiPSC-derived MSC-like cells (11C) shows the engraftment of the implants, Alcian blue stain (11D-11F) demonstrates the glycosaminoglycan production of chondrogenic differentiated cells (11F), and immunohistochemistry (11G-11I) of the implants shows the production of the Collagen type II. Arrowheads demonstrate the borders of the defect and the complete arrows show the residual of scaffold. Scale bar is equal to 500 microns. hiPSC grown on the scaffold only (11A, 11D, and 11G) was a control.

### DETAILED DESCRIPTION

Provided herein are methods for differentiating stem cells into a mesenchymal lineage cells without embryoid body formation as well as methods for differentiating stem cells into chondrogenic cells without embryoid body formation. These methods provide for a better controlled, more efficient, and simpler mesenchymal and/or chondrogenic differentiation process. Term definitions used in the specification and claims to describe the subjects of the present disclosure are as follows.

### Definitions

As used herein, “cell,” “cell line,” and “cell culture” include progeny. It is also understood that all progeny may not be precisely identical in DNA content, due to deliberate or inadvertent mutations. Variant progeny that have the same function or biological property, as screened for in the originally transformed cell, are included.

As used herein, “chondrogenic cell” refers to a chondrocyte at any stage of maturation and may express one or more of the following markers: annexin VI, Col2a1(IIa), beta1 Integrin (CD29), N-cadherin (Ncad), N-cam (Ncam1), tenascin C (Tnc), sox9, CEP-68, MMP13 (matrix metalloproteinase-13), Matrilin-1, Col9, 11-fibrau, Syndecan-3, Col2a1(IIb), and aggrecan.

As used herein, “chondrocyte” refers to a cell that produces one or more of the components of cartilage, including collagen and proteoglycans.

As used herein, “chondroblast” refers to an immature chondrocyte.

As used herein, “chondrogenic differentiated stem cell” refers to a “chondrogenic cell.”

As used herein, “composition” refers to a combination of active agent and at least one other compound or molecule, inert (for example, a detectable agent or label) or active, such as an adjuvant.

As used herein, “control” is an alternative subject or sample used in an experiment for comparison purpose and included to minimize or distinguish the effect of variables other than an independent variable.

As used herein, “positive control” refers to a “control” that is designed to produce the desired result, provided that all reagents are functioning properly and that the experiment is properly conducted.

As used herein, “negative control” refers to a “control” that is designed to produce no effect or result, provided that all reagents are functioning properly and that the experiment is properly conducted. Other terms that are interchangeable with “negative control” include “sham,” “placebo,” and “mock.”

As used herein, “culturing” refers to maintaining cells under conditions in which they can proliferate and avoid senescence as a group of cells. “Culturing” can also include conditions in which the cells also or alternatively differentiate.

As used herein, “differentially expressed,” refers to the differential production of RNA, including but not limited to mRNA, tRNA, miRNA, siRNA, snRNA, and piRNA transcribed from a gene or regulatory region of a genome or the protein product encoded by a gene as compared to the level of production of RNA by the same gene or regulator region in a normal or a control cell. In another context, “differentially expressed,” also refers to nucleotide sequences or proteins in a cell or tissue which have different temporal and/or spatial expression profiles as compared to a normal or control cell.

As used herein, “overexpressed” or “overexpression” refers to an increased expression level of an RNA or protein product encoded by a gene as compared to the level of expression of the RNA or protein product in a normal or control cell.

As used herein, “underexpressed” or “underexpression” refers to decreased expression level of an RNA or protein product encoded by a gene as compared to the level of expression of the RNA or protein product in a normal or control cell.

As used herein, “differentiate” or “differentiation,” refers to the process by which precursor or progenitor cells (i.e., chondrogenic progenitor cells) differentiate into specific cell types, e.g., chondrogenic cells.

As used herein, “effective amount” is an amount sufficient to effect beneficial or desired biological, emotional, medical, or clinical response of a cell, tissue, system, animal, or human. An effective amount can be administered in one or more administrations, applications, or dosages. The term also includes, within its scope, amounts effective to enhance normal physiological function.

As used herein, “expansion” or “expandeds” in the context of cells, refers to an increase in the number of a characteristic cell type, or cell types, from an initial population of cells, which may or may not be identical. The initial cells used for expansion need not be the same as the cells generated from expansion. For instance, the expanded cells may be produced by ex vivo or in vitro growth and differentiation of the initial population of cells.

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins.

As used herein, “induced pluripotent stem cell” or “iPS cell” refers to a cell capable of differentiating into multiple cell types that is artificially derived (not naturally derived) from a non-pluripotent cell.

As used herein, “integration free iPS cell” refers to an iPS cell that does not contain an exogenous transgene integrated into the genome of the non-pluripotent cell.

As used herein, “isolated” means separated from constituents, cellular and otherwise, in which the polynucleotide, peptide, polypeptide, protein, antibody, or fragments thereof, are normally associated with in nature. A non-naturally occurring polynucleotide, peptide, polypeptide, protein, antibody, or fragments thereof, do not require “isolation” to distinguish it from its naturally occurring counterpart.

As used herein, “concentrated” refers to a molecule, including but not limited to a polynucleotide, peptide, polypeptide, protein, antibody, or fragments thereof, that is distinguishable from its naturally occurring counterpart in that the concentration or number of molecules per volume is greater than that of its naturally occurring counterpart.

As used herein, “diluted” refers to a molecule, including but not limited to a polynucleotide, peptide, polypeptide, protein, antibody, or fragments thereof, that is distinguishable from its naturally occurring counterpart in that the concentration or number of molecules per volume is less than that of its naturally occurring counterpart.

As used herein, “separated” refers to the state of being physically divided from the original source or population such that the separated compound, agent, particle, or molecule can no longer be considered part of the original source or population.

As used herein, “embryoid body” refers to a three-dimensional spheroid aggregate of pluripotent stem cells, including but not limited to embryonic stem cells derived from the blastocyst stage of embryos from mammalian sources. An embryoid body can be formed from embryonic stem cells derived through any technique generally known in the art, including but not limited to somatic cell nuclear transfer or the reprogramming of somatic cells to yield induced pluripotent stem cells.

As used herein, “mammal,” for the purposes of treatments, refers to any animal classified as a mammal, including human, domestic and farm animals, nonhuman primates, and zoo, sports, or pet animals, such as, but not limited to, dogs, horses, cats, and cows.

As used herein, “mesenchymal stem cell” or “MSC” refers herein to a multipotent cell capable of differentiating into cells that compose adipose, bone, cartilage, and muscle tissue.

As used herein, “mesenchymal lineage cell” refers to a multipotent cell that is capable of differentiating into one or more cells that compose adipose, bone, cartilage, and muscle tissue, and, may or may not express the surface protein markers that are commonly associated with mesenchymal stem cells.

As used herein, “mesenchymal cell population” refers to a population of cells that comprises one or more of mesenchymal stem cells, mesenchymal lineage cells, osteoblasts, adipocytes, and chondrocytes. In such a population, the osteoblasts, adipocytes, and chondrocytes can be immature, mature, or a combination thereof.

As used herein, “mesenchymal stem cell-like” refers to cells derived from a stem cell population that are morphologically similar to mesenchymal stem cells and express cell surface markers typical of mesenchymal stem cells. Although mesenchymal stem cell-like cells may have some overlapping characteristics (e.g. similar morphology) with mesenchymal stem cells, they are not identical to mesenchymal stem cells for at least the fact that mesenchymal stem cell-like cells are not directly derived from bone marrow aspirate. Mesenchymal stem cells are derived directly from bone marrow aspirate.

As used herein, “stem cell” refers to any self-renewing totipotent, pluripotent cell or multipotent cell or progenitor cell or precursor cell that is capable of differentiating into multiple cell types.

As used herein, “totipotent” refers cells that can differentiate and give rise to all cells types in an organism, plus the extraembryoinc, or placental, cells.

As used herein, “pluripotent” refers to cells that can differentiate and give rise to all of the cell types that make up an organism, except for the extraembryonic, or placental, cells.

As used herein, “multipotent” refers to cells that can develop into more than one cell type, but are more limited than pluripotent cells in the cell types that they can develop into.

As used interchangeably herein, “subject,” “individual,” or “patient” refers to a vertebrate organism.

As used herein, “substantially pure cell population” refers to a population of cells having a specified cell marker characteristic and differentiation potential that is about 50%, preferably about 75-80%, more preferably about 85-90%, and most preferably at least about 95% of the cells making up the total cell population. Thus, a “substantially pure cell population” refers to a population of cells that contain fewer than about 50%, preferably fewer than about 20-25%, more preferably fewer than about 10-15%, and most preferably fewer than about 5% of cells that do not display a specified marker characteristic and differentiation potential under designated assay conditions.

As used herein, “pre-differentiation” refers to the process by which precursor or progenitor cells (e.g., pluripotent stem cells) differentiate into intermediate cell types, e.g., mesenchymal stem cells, which have the potential to differentiate further to final effector cells (e.g. chondrocytes).

As used herein, “hydrogel” refers to a non-fluid colloidal network or polymer network that is expanded throughout its whole volume by water.

As used herein, “matrix” refers to material in animals or plants, in which more specialized structures are embedded.

As used herein, “biocompatible” or “biocompatibility” refers to the ability of a material to be used by a patient without eliciting an adverse or otherwise inappropriate host response in the patient to the material or a derivative thereof, such as a metabolite, as compared to the host response in a normal or control patient.

As used herein, “biodegradable” refers to the ability of a material or compound to be decomposed by bacteria or other living organisms or organic processes.

As used herein, “therapeutic” refers to treating, healing, and/or ameliorating a disease, disorder, condition, or side effect, or to decreasing in the rate of advancement of a disease, disorder, condition, or side effect. The term also includes within its scope enhancing normal physiological function, pallative treatment, and partial remediation of a disease, disorder, condition or side effect.

The terms “treating” and “treatment” as used herein refer generally to obtaining a desired pharmacological and/or physiological effect. The effect may be prophylactic in terms of preventing or partially preventing a disease, symptom or condition thereof such as of OA, and/or may be therapeutic in terms of a partial or complete cure of a disease, condition, symptom, or adverse effect attributed to the disease. The term “treatment” as used herein covers any treatment of OA or other cartilage disorder in a mammal, particularly a human, and includes: (a) preventing the disease from occurring in a subject which may be predisposed to the disease but has not yet been diagnosed as having it; (b) inhibiting the disease, i.e., arresting its development; or (c) relieving the disease, i.e., mitigating or ameliorating the disease and/or its symptoms or conditions. The term “treatment” as used herein refers to both therapeutic treatment and prophylactic or preventative measures. Those in need of treatment include those already with the disorder as well as those in which the disorder is to be prevented.

As used herein, “preventative” refers to hindering or stopping a disease or condition before it occurs, even if undiagnosed, or while the disease or condition is still in the sub-clinical phase.

As used herein, “active agent” refers to a substance, compound, or molecule, which is biologically active or otherwise induces a biological or physiological effect on a subject to which it is administered to.

As used herein, “teratoma” refers to an encapsulated tumor with tissue or organ components resembling normal derivatives of more than one germ layer.

As used herein, “pharmaceutically acceptable carrier” refers to diluent, adjuvant, excipient, or vehicle with which an active agent, chondrocytes of the present disclosure, or composition containing chondrocytes of the present disclosure is administered in conjunction with and that is approved by a regulatory agency of the Federal or a state government or listed in the U.S. Pharmacopeia or other generally recognized pharmacopeia for use in animals and/or humans.

Unless otherwise defined herein, all technical and scientific terms used herein have the same meaning as commonly understood by one of ordinary skill in the art.

### DISCUSSION

As shown in FIG. 1, current methods of iPSC differentiation require the formation of embryoid bodies. See also Koyama et al., Stem Cells Dev. 22(1):102-113. Generally, embryoid bodies are formed and maintained in suspension. This is followed by mesenchymal stem/stromal cell outgrowth from the formed embryoid bodies. Finally, differentiation down a desired pathway occurs after culturing in differentiation media. More specifically, on about day 1, embryoid bodies are formed. After culturing for about 5 days (days 1-5), outgrowth of mesenchymal stem/stromal cells from the embryoid bodies is induced. On about day 14 of the process, cells are collected and filtered so as to separate out the fibroblast-like cells from the other types of cells developed from ectodermal, mesodermal, and endodermal lineages present in the embryoid body outgrowth. From about Day 14 to about Day 35, the fibroblast-like cells are cultured as a monolayer differentiated into mesenchymal stem cell (MSC)-like cells. At day 35 the MSC-like cells are pelleted to form a cell pellet. The cell pellet is further cultured using a 3D pellet culturing method as described in Koyama et al. After about 2-3 weeks of pellet culture (about day 56), cells in the pellet exhibit a morphology associated with the desired differentiated cell lineage. For example, as shown in FIG. 2, the cells pelleted and cultured in chondrogenic differentiation media show a spherical morphology typical of chondrocytes and are surrounded by extracellular matrix containing acidic proteoglycans.

In contrast, the methods of the present disclosure produce cells in a mesenchymal lineage, e.g. chondrocytes, from differentiation of toti-, pluri-, or multi-potent stem cells, e.g. iPSCs, and more particularly, hiPSCs, without the formation of embryoid bodies. More specifically, the methods described herein involve direct differentiation of mesenchymal stem cells (MSCs) under specific cell culture conditions followed by chondrogenic differentiation. The methods of the present disclosure are more efficient than current methods at least for the reason that they bypass differentiation into undesired cell types. Additionally, the methods, cells, and compositions provided herein provide a framework for rational experimental and clinical trial design, as well as a more uniform and reproducible approach for cartilage regeneration therapeutics. These methods can be applied to other pluripotent stem cells, such as human embryonic stem cells (hESC), and can be adapted for differentiation pathways other than those for chondrogenic lineages.

In general, first the stem cells are cultured in a suitable stem cell medium as adherent cells. Stem cell media refers to media suitable for culturing cells while maintaining the toti-, pluri-, or multi-potency of the stem cells. This is followed by pre-differentiation of the cells. Pre-differentiation of the stem cells is induced by culturing the stem cells in a suitable MSC media for about 3 to 7 days. This produces toti- pluri-, or multi-potent stem cell-MSC like cells. MSC media is media suitable for differentiating cells down or maintaining cells in a MSC lineage. The toti- pluri-, or multi-potent stem cell-derived MSC-like cells are then differentiated into any desired mesenchymal lineage cell by culturing in a suitable differentiation medium using generally known culture methods.

With the general method in mind, attention is directed to FIG. 2, which shows one embodiment of an embryoid body-free method of generating chondrocytes from induced pluripotent stem cells. In one embodiment, the method begins by culturing undifferentiated iPSCs in a suitable iPSC media with or without a feeder layer for about 1 to about 5 days. In embodiments where iPSCs are cultured without a feeder layer, the iPSC media is media suitable to sustain iPSC culture without a feeder layer. Suitable medias include, but are not limited to, E8 media, mTeSR™1 media, mTeSR™2 media, Knockout™ Dulbecco's modified essential media (DMEM) supplemented with Knockout™ serum replacement, Knockout™ serum replacement (SR) XenoFree media, Knockout™ serum replacement (SR) XenoFree media supplemented with Knockout™ SR growth factor cocktail, ReproFF2™, ReproCell, and PluriSTEM™ media, NutriStem XF/FF™ media. In embodiments where a feeder layer is used, suitable medias include, but are not limited to, KnockOut™DMEM/F-12.

In some embodiments, the iPSCs are cultured in culture vessels that have a surface treated or coated with a suitable compound or compounds to enhance, maintain, or otherwise modify cell growth, differentiation, and/or attachment of the iPSCs. Suitable compounds include, but are not limited to extracellular matrix (ECM) components such as collagen, whole ECM (with “cellular ECM” or without cells “acellular ECM”), self-assembling peptide scaffolds, polylysine, gelatin, synthetic nanofibers, and combinations thereof.

The iPSCs can be derived from any suitable source according to any suitable methods. In one embodiment, suitable reprogramming factors are delivered to donor somatic cells using suitable viral or nonviral methods, as generally known in the art. In some embodiments, the reprogramming factors are selected from Oct4, Sox2, Lin28, Nanog or combinations thereof. In other embodiments, the reprograming factors are selected from Oct4 (Pou5f1), Sox2, cMyc, and Klf4. The somatic donor cell can be any somatic cell suitable for reprogramming into toti-, pluri-, or multi-potent stem cells. In some embodiments, the somatic donor cells are adipose stem cells, fibroblasts, blood, or epithelial cells. The donor somatic cells can be derived from any organism. In some embodiments, the donor somatic cells are derived from a non-mammalian species. In other embodiments, the donor somatic cells are derived from a mammalian species. For some embodiments, the somatic cells are derived from a human, in which the derived iPSCs are referred to as hiPSCs. While in some embodiments, the somatic donor cells are autologous, in others the somatic donor cells are non-autologous.

Next, the iPSCs are directly differentiated into MSC-like cells to form iPSC-derived MSC-like cells. This step is shown as day 1 in FIG. 2. To directly differentiate iPSCs, undifferentiated iPSCs are cultured to a suitable confluency, at which point the iPSC media is replaced with MSC media. In some embodiments, the iPSC media is replaced with MSC media when the confluency of the iPSCs reaches about 40% to about 100%. In other embodiments, the iPSC media is replaced with MSC media when the confluency of the iPSCs reaches about 50%. The confluence at which the iPSC is replaced with MSC media will depend on, among other things, the type of iPSC cell used. MSC media is suitable for culturing MSCs. In one embodiment, the MSC media is high glucose DMEM supplemented with 10% fetal bovine serum, 100 U/mL Penicillin and 100 mg/mL Streptomycin. Other suitable medias for MSC culture are generally known in the art. The iPSCs are grown without sub-culturing in MSC media for about 5 days. During this time period, MSC media is replaced with new MSC media at least once. In some embodiments, the MSC media is replaced at least once every day.

On about day 5 of culturing in MSC media, the pre-differentiating cells are detached from the coated surface of the culture vessel (feeder-free embodiments) or separated from the feeder layer of cells using standard sub-culturing techniques. The sub-cultured pre-differentiating cells are then cultured in suitable culture vessels. In one embodiment, the culture vessels are uncoated polystyrene culture flasks. The pre-differentiating cells are allowed to proliferate until they reach a suitable confluency, at which point they are sub-cultured. In some embodiments, the proliferating pre-differentiating cells are about 60% to about 95% confluent at the time of sub-culturing. In other embodiments, the proliferating pre-differentiating cells are about 90% confluent at the time of sub-culturing. The confluency at which the proliferating, pre-differentiating cells are sub-cultured will depend on the type of iPSC used, among other things.

After this initial subculture, in some embodiments the proliferating pre-differentiating cells are sub-cultured at a ratio ranging from of about 1:1 to about 1:5 until about passage 1 (P1) to about passage 5 (P5). In one embodiment, the proliferating pre-differentiating cells are expanded out to passage 4 (P4). For hiPSCs, P4 occurs on about Day 21 (See FIG. 2). In another embodiment, the proliferating pre-differentiating cells are sub-cultured at a ratio of about 1:3. At this point, the cells are considered iPSC-derived MSC-like cells. In some embodiments, the iPSC-derived MSC-like cells express MSC markers such as, but not limited to, CD105, CD73 and CD90. In other embodiments, the iPSC-derived MSC-like cells do not express CD45, CD34, CD14, CD11b, CD 19, or HLA-DR.

iPSC-derived MSC-like cells are further differentiated down a desired differentiation pathway by culturing in differentiation media. In some embodiments, the iPSC-derived MSC-like cells are differentiated down a chondrogenic pathway. For these embodiments, the iPSC-derived MSC-like cells are differentiated using suitable methods of chondrogenic differentiation. For other embodiments, a 3D, high-density pellet culture procedure is employed to induce chondrogenic differentiation. In some embodiments, about 1×10⁴ to about 1×10⁶ P4 iPSC-derived MSC-like cells are detached from the culture vessel using standard detachment procedures and pelleted using centrifugation. In other embodiments, about 2.5×10⁵ P4 iPSC-derived MSC-like cells are pelleted.

The pelleted cells are then cultured in a suitable chondrogenic differentiation media. In one embodiment, the chondrogenic differentiation media contains high glucose DMEM, about 100 U/mL penicillin, about 100 μg/mL streptomycin, about 10% L-glutamine, about 50 μg/mL L-ascorbic acid 2-phosphate sequimagnesium, about 100 μg/mL minimal essential medium sodium pyrovate, about 40 μg/mL L-proline, about 100 nM dexamethasone, 10 ng/mL TGF-β3, ITS+Premix at a final concentration of about 5.5 μg/mL transferrin, about 10 μg/mL bovine insulin, about 5 μg/mL sodium selenite, about 4.7 μg/mL linoleic acid, and about 500 μg/mL bovine serum albumin.

The pellets are then cultured in chondrogenic differentiation media for about 18 to 24 days. As shown in FIG. 2, in some embodiments, the pellets are cultured in chondrogenic differentiation media for about 21 days to form chondrocytes. In other embodiments, after 18-24 days in chondrogenic differentiation media, the chondrocytes express cartilage markers. In one embodiment, the chondrocytes have increased expression Col2A1, Sox-9, and/or aggrecan as compared to a non-differentiated cell.

Also within the scope of the present disclosure are compositions containing chondrocytes produced according to the methods described herein. In some embodiments the compositions also contain a matrix. In other embodiments, the matrix includes natural materials, such as polysaccharides, including but not limited to starch, alginate, chitin/chitosan, and/or hyaluronic acid derivatives, and/or proteins, including but not limited to soy, collagen, hyaluronan, fibrin gels, and silk, cellular ECM and/or acullular ECM. In some embodiments, the matrix includes synthetic polymers, such as poly(lactic acid), poly(glycolic acid), poly(3-caprolactone), poly (hydroxyl butyrate), poly(lactic-co-glycolic) acid, self-assembling peptides, and/or carbon nanotubes. In further embodiments, the matrix is biocompatible. In one embodiment, the matrix is a biodegradable matrix. The components of the matrix can be autologous or non-autologous.

In some embodiments the compositions containing chondrocytes also contain a hydrogel. The hydrogel contains natural polymers, synthetic polymers, or combinations thereof. Suitable natural polymers include, but are not limited to, collagen, alginate, hyaluronic acid, fibrin, agarose, and chitosan. Suitable synthetic hydrogels include, but are not limited to, poly(hydroxyethyl methacrylate), poly(ethylene glycol), poly(vinyl alcohol), poly (acrylic acid), poly(methacrylic acid), polyacrylamide, poly (diethylaminoethyl methacrylate), poly(ethylene oxide), and poly(dimetholaminoethyl methacrylate), poly(N-iospropylacrylamide), poly(lactic acid), poly(glycolic acid), poly(3-caprolactone), and derivatives thereof. In some embodiments, the hydrogel is homopolymeric, co-polymeric, or multipolymer interpenetrating polymeric.

The hydrogel can be amorphous, semicrystalline, which is a complex mixture of amorphous and crystalline phases, or crystalline. The hydrogel can be cross-linked or exist as a physical network. The polymers within a cross-linked hydrogel are permanently linked, whereas the polymers within a physical network are joined by transient linkages that arise either from polymer chain entanglements or physical interactions such as ionic interactions, hydrogen bonding, or hydrophobic interactions. The hydrogel can be nonionic, ionic (cationic or anionic), ampholytic, or zwitterionic. In some embodiments, the hydrogel is biocompatible. In other embodiments, the hydrogel is biodegradable.

In further embodiments, the compositions containing chondrocytes produced according to the methods of the present disclosure also contain at least one active agent present at a dose effective for treatment or prevention of a condition. In some embodiments containing a matrix, the active agent is contained within or associated with the matrix. In some embodiments containing a hydrogel, the active agent is contained within the hydrogel. Suitable active agents include, but are not limited to, immunomodulators, cytokines, chemokines, growth factors, analgesics, anesthetics, anti-inflammatory agents (including steroids and nonsteroidal anti-inflammatory agents), antihistamines, peptides, proteins, antibodies and fragments thereof, plasmids, naked DNA, viral vectors, RNA (including, but not limited to, miRNA, siRNA, piRNA, and short hairpin RNA), amino acids, aptamers, anti-infective agents, antineoplastic agents, blood derivatives, blood formation agents, coagulation agents, thrombosis agents, diagnostic agents (including, but not limited to, contrast agents and fluorescent agents), radioactive agents, antipyretics, vitamins, enzymes, and other cell types.

In some embodiments, the active agent and/or the compositions containing chondrocytes produced by the methods described herein also include a pharmaceutically acceptable carrier. In some embodiments the pharmaceutical carriers are liquids, such as water and/or oils, including those of petroleum, animal, vegetable, or synthetic origin, such as peanut oil, soybean oil, mineral oil, sesame oil, and the like. In other embodiments, the pharmaceutically acceptable carrier is gum acacia, gelatin, agar, agarose, collagen, starch paste, talc, keratin, colloidal silica, urea, and the like. In other embodiments, the pharmaceutically acceptable carrier is saline solution, aqueous dextrose solution, or glycerol solutions. These embodiments are particularly useful for injectable compositions. In further embodiments, the pharmaceutically acceptable carrier includes an excipient, such as glucose, lactose, sucrose, glycerol monostearate, sodium chloride, glycerol, propylene, glycol, water, ethanol and combinations thereof. In some embodiments, the compositions can contain an amount of wetting or emulsifying agents, or pH buffering agents. The present compositions my take the form of solutions, emulsions, sustained-release formulations, or any other form suitable for use.

Chondrocytes and compositions containing chondrocytes produced by the methods described herein are useful for the prevention or treatment of OA, traumatic cartilage defects and other cartilage injuries. In one embodiment, the chondrocytes described herein or compositions containing the chondrocytes described herein are implanted to replace damaged cartilage or chondral lesions. For example, during joint surgery, the chondral lesions can be removed from the articular surface of the joint forming cavities on the surface of the articular surface of the joint being treated. Chondrocytes or a composition containing chondrocytes produced by the methods described herein can be implanted into the cavities formed by the removal of chondral lesions.

In other embodiments, the chondrocytes or compositions containing chondrocytes produced by the methods described herein can be administered to a patient via intra-articular (IA) or intravenous (IV) injection. In some embodiments, the chondrocytes of compositions containing chondrocytes produced via the methods described herein are administered to a patient via IA injection to a joint. The joint can be any joint in the body in need of treatment, including but not limited to, knee, shoulder, hip, ankle, sacroiliac, lumbar, elbow, wrist, or cervical. In other embodiments, the chondrocytes and compositions containing chondrocytes produced by the methods described herein can be used for in vitro cartilage tissue generation.

Without further elaboration, it is believed that one skilled in the art can, based on the description herein, utilize the present disclosure to its fullest extent. It is emphasized that the embodiments of the present disclosure, particularly any “preferred” embodiments, are merely possible examples of the implementations, merely set forth for a clear understanding of the principles of the disclosure. Many variations and modifications may be made to the disclosed embodiment(s) of the disclosure without departing substantially from the spirit and principles of the disclosure. All such modifications and variations are within the scope of this disclosure.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of molecular biology, microbiology, nanotechnology, organic chemistry, biochemistry, botany and the like, which are within the skill of the art. Such techniques are explained fully in the literature.

### EXAMPLES

The following examples are put forth so as to provide those of ordinary skill in the art with a complete disclosure and description of how to perform the methods and use the compositions and compounds disclosed and claimed herein. The specific examples below are to be construed as merely illustrative and not limitative of the remainder of the disclosure in any way. Efforts have been made to ensure accuracy with respect to numbers (e.g. amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Unless described otherwise, data analyses were performed as follows. Gene expression levels of original hiPSC, intermediate hiPSC-derived MSC-like cells, and chondrogenic pellets at days 0 and 14 of chondrogenic differentiation were compared with an analysis of variance (ANOVA). A Bonferroni correction was applied for comparisons. An analysis of variance (ANOVA) with a Bonferroni correction were used to compare MRI T2 relaxation times of each groups overtime and also the T2 relaxation times of scaffold only group were compared with hiPSC-derived MSC-like and hiPSC chondrogenic differentiated cell pellets groups. All statistical analysis were performed using GraphPad Prism 6 statistical software (GraphPad Software Inc. CA, USA).

### Example 1

### Culture of Undifferentiated hiPS Cells

hiPS cell lines from human adipose derived stem cells were derived, using the minicircle reprogramming technique. An iPS-ASC3 cell line was developed in the Wu lab (Department of medicine and radiology, Stanford medical school) according to the methods described in Jia et al, A nonviral minicircle vector for deriving human iPS cells, Nature Methods, Vol. 7, No. 3, 197-199 (2010). For generation of hiPS cell lines from adult human fibroblasts, a codon-optimized minicircle plasmid was used (Diecke et al. in press Methods in Molecular Biology). The iPS cells were cultured as undifferentiated, pluripotent stem cells in 10 cm (BD Falcon, Sparks, Md., USA) petri-dishes coated with 1% matrigel (BD Matrigel™Basement Membrane Matrix). Cells were grown at 37° C. in a 5% CO₂ atmosphere in mTeSR1™ media (Stem Cell™ Technologies, Vancouver, BC, Canada) in a feeder layer culture condition. The medium of the cells was changed at least once a day and every 4-5 days, cells were sub-cultured at about a 1:6 ratio. FIGS. 5A and 5B show a microscopic image of iPS cells (5A) cultured without a feeder layer and (5B) cultured with MSC media.

### Example 2

### Pre-Differentiation of iPS Cells into iPS-MSC Like Stem Cells

The undifferentiated iPS cells described above were cultured in 10 cm petri-dishes (BD Falcon, Sparks, Md., USA) coated with 1% Matrigel (BD Matrigel™ Basement Membrane Matrix) to reach 40-50 percent confluency. The mTeSR1™ media (Stem Cell™ Technologies, Vancouver, BC, Canada) was changed to high glucose Dulbecco's Modified Eagle Medium (DMEM) supplemented with 10% fetal bovine serum (FBS) and 100 units/mL of penicillin and 100 mg/mL of streptomycin (all Invitrogen, Carlsbad, Calif., USA). Media was changed every day for 5 days. On about Day 5, cells were detached using about 5% trypsin EDTA (Invitrogen, Carlsbad, Calif., USA) and cultured in polystyrene culture flasks (Fisher Scientific Company, Pittsburgh, Pa., USA). Media was changed every day for the first 5 days and then every other day until cells were 90% confluent. Cells were sub-cultured at a ratio of approximately 1:3. The cells were cultured until passage 5, and these cells were used for the rest of the experiments. FIGS. 5C and 5D show a microscopic image of iPS cells (5C) as a subculture of pre-differentiated cells transferred to an uncoated culture flask, and (3D) iPSC-MSC-like stem cells.

### Example 3

### Chondrogenic Differentiation

At passage 4, pre-differentiated iPS cells were detached using 5% tryspin EDTA (Invitrogen) and counted. 2.5×10⁵ cells were resuspended in 0.5 ml of serum-free chondrogenic differentiation media (high glucose DMEM, 100 U/ml penicillin, 100 μg/ml streptomycin (Gibco), 10% L-Glutamine (Gibco), 50 μg/ml L-ascorbic acid 2-phosphate sequimagnesium (Sigma), 100 μg/ml MEM sodium pyrovate (Gibco), 40 μg/ml L-proline (Sigma), 100 nM dexamethasone (Sigma), ITS+Premix (final concentration: 5.5 μg/ml transferring, 10 μg/ml bovine insulin, 5 μg/ml sodium selenite, 4.7 μg/ml linoleic acid, and 500 μg/ml bovine serum albumin (BD Bioscience, Franklin Lakes, N.J.)), and 10 ng/ml TGF-β3 (R&D Systems, Minneapolis, Minn.)) and centrifuged again at 1000 rpm for 5 minutes and incubated at about 37° C. in about a 5% CO₂ atmosphere. Cell pellet medium was changed every other day for 14 days. Pellets were harvested at about day 0, 7, and 14 of chondrogenic differentiation for gene expression evaluation. FIG. 7 shows Col2A1 gene expression level in chondrogenic differentiated stem cells over time.

At about day 14, one pellet was fixed in formalin, and dehydrated through graded alcohol washes (about 70%, about 95% and about 100%) and xylene. The pellets were then embedded in paraffin and sliced into 5 μm thick tissue slices on glass slides. The slides were de-waxed and used for stains such as H&E, Alcian blue staining and immunohistochemistry. See FIGS. 8A through 8F, which show a microscopic image of an H&E stain of chondrogenic differentiated iPSC-derived MSC-like cells (8A & 8B), an Alcian blue stain that demonstrates the glycosaminoglycan production by the chondrogenic differentiated iPSC-derived MSC-like cells (8C & 8D), and an immunohistochemical analysis showing the production of the Collagen type II by these cells (8E & 8F).

### Example 4

### Pluripotency Evaluation and Teratoma Formation of hiPSC

### Materials and Methods

This study was approved by the Committee on Human Research and the Stem Cell Research Oversight (SCRO) Committee at our institution. hiPSC were developed using established procedures as described in Jia et al., Nat. Methods, 7(3):197-199. The undifferentiated hiPSC were cultured in mTeSR1™ media (Stem Cell™ Technologies, Vancouver, BC, Canada) in 10 cm petri dishes (BD Falcon, Sparks, Md., USA) coated with about 1% matrigel (BD Matrigel™ Basement Membrane Matrix) at 37° C. in a 5% CO₂ atmosphere. This media was changed every day and cells were sub-cultured about every 4-5 days.

Pluripotency of the hiPSC were evaluated by immune-staining for pluripotency markers OCT4, SOX2, NANOG and TRA-1-60. Subcutaneous injection of hiPSC was performed to evaluate the teratoma formation of the hiPSC in severe combined immunodeficient (SCID) mice. As shown in FIGS. 3A-3F and 4A-4C, the resulting teratomas were sectioned and stained to explore all three germ layers' differentiation, including endoderm, mesoderm, and ectoderm.

### Results

The pluripotency of the generated hiPSC was confirmed by positive immunofluorescence staining for NANOG, OCT4, SOX2 and TRA-1-60 (pluripotency markers) (FIGS. 3A, 3B, 3E, and 3F). In addition, H&E stains of hiPSC-derived teratomas showed all three germ layers differentiation, including ectoderm, mesoderm, and endoderm (FIGS. 4A-4C).

### Example 5

### Direct Differentiation of hiPSC into MSC-Like Cells

### Materials and Methods

hiPSC-derived MSC-like cells were generated as described in Example 4 and as shown in FIGS. 3A-3H and 4A-4C. Undifferentiated hiPSC were cultured to reach about 50% confluency. Then, the mTeSR1™ media was changed to typical hMSC culture media, i.e. high glucose Dulbecco's Modified Eagle Medium (DMEM, Invitrogen, Carlsbad, Calif., USA) with about 10% stem cell qualified fetal bovine serum (FBS, Invitrogen, Carlsbad, Calif., USA), 100 units/mL of Penicillin, and 100 mg/mL of Streptomycin (Invitrogen, Carlsbad, Calif., USA). The DMEM high glucose media was changed every day for about 5 days. On about day 5, cells were detached from the matrigel-coated petri dishes using about 5% Trypsin/EDTA (Invitrogen, Carlsbad, Calif., USA) and cultured in uncoated polysterene culture flasks (Fisher Scientific Company, Pittsburgh, Pa., USA). Media was changed every other day until the cells reached about 90% confluency. Then, the cells were sub-cultured at a ratio of 1:3 until passage 4 (P4). The cell morphology of original hiPSC (FIG. 5, Day 0), and hiPSC-derived MSC-like (FIG. 5, Day 1-21) were observed by phase contrast imaging over time (FIG. 5).

### Example 6

### Phenotyping of hiPSC and hiPSC-Derived MSC-Like Cells

### Materials and Methods

In order to characterize the phenotypes of the differentiated hiPSC-derived MSC-like cells, triplicate samples of hiPSC and hiPSC-derived MSC-like cells underwent flow cytometry analyses on a BD FACS Canto II flow cytometer. Compensation was set using BD Comp Beads. The cells were tested for MSC markers according to the international society for cell therapy (ISCT) criteria (Dominici et al., Cytotherapy. 2006; 8(4): 315-317), which included presence of CD105, CD73 and CD90, as well as lack of CD45, CD34, CD14 or CD11b, CD19 and HLA-DR surface molecules. Data were quantified using BD FACS Diva software (BD Biosciences, San Jose, Calif., USA) and Flowjo™ data analysis package (http://www.Treestar.com).

### Results

After changing mTeSR media to hMSC media, cells started to differentiate and spread out from their colonies. Although initially, a heterogeneous morphologic cell population developed, more than about 90% of the total cell population acquired a fibroblast-like morphology by passage 4, which matched the typical morphology of hMSC (FIG. 5 Day 21). In order to determine phenotypes and genotypes of hiPSC-derived MSC-like cells, standard microscopy and flow cytometry analyses were performed. The flow cytometry analysis showed that the majority of the cells were positive for the hMSC markers CD105 (>about 93%), CD73 (>about 96%), and CD90 (>about 95%), and negative for CD45, CD34, CD14 or CD11b, CD19, and HLA-DR surface molecules (>about 95%), See (FIGS. 6A-6L) and Table 1.

TABLE 1

| | | | | CD34/11b/19/ |
|---|---|---|---|---|
| | CD73 | CD90 | CD105 | 45/HLA-DR |
| hiPSC | 1.06 ± 1.41 | 67.4 ± 0.05 | 0.3 ± 0.1 | 2.5 ± 1.73 |
| hiPSC- | 96.56 ± 0.56 | 95.23 ± 0.32 | 93.83 ± 0.58 | 5 ± .07 |
| derived | | | | |
| MSC-like | | | | |

### Example 7

### In Vitro Chondrogenic Differentiation of hiPSC-Derived MSC-Like Cells

### Materials and Methods

In order to evaluate the chondrogenic potential of the hiPSC-derived MSC-like cells in vitro, the cells were detached from culture flasks using about 5% Tryspin/EDTA and underwent chondrogenic differentiation in a 3D, high-density pellet culture using protocols as described in Nejadnik et al., PloS one. 2012; 7(12):e49971. In brief, centrifuged pellets of 2.5×10⁵ hiPSC-derived MSC-like cells were incubated in about 5% CO₂ at about 37° C. and in about 0.5 mL of serum-free chondrogenic differentiation media, containing high glucose DMEM, 100 U/mL penicillin, 100 μg/mL streptomycin, 10% L-Glutamine (Gibco), 50 μg/mL L-ascorbic acid 2-phosphate sequimagnesium (Sigma), 100 μg/mL MEM sodium pyrovate (Gibco), 40 μg/mL L-proline (Sigma), 100 nM dexamethasone (Sigma), ITS+Premix final concentration: 5.5 μg/mL transferring, 10 μg/mL bovine insulin, 5 μg/mL sodium selenite, 4.7 μg/mL linoleic acid, and 500 μg/mL bovine serum albumin (BD Bioscience, Franklin Lakes, N.J.), and supplemented with 10 ng/mL TGF-β3 (R&D Systems, Minneapolis, Minn.). The chondrogenic medium was changed every other day for about 21 days. Pellets were harvested at about day 0, 7, and 14 of chondrogenic differentiation for gene expression analysis and about day 21 for standard histopathology, immunohistochemistry.

Quantitative real-time PCR (qPCR) was used to further confirm chondrogenic differentiation of the hiPSC-derived MSC-like cells. Gene expression levels of the differentiated cells were assessed for hyaline cartilage markers collagen type II (Col2A1), SRY (sex determining region Y)-box 9 (SOX9), and Aggrecan (ACAN), and the control marker of Glyceraldehyde 3-phosphate dehydrogenase (GAPDH). In brief, the total cellular RNA was extracted from each sample with the QIAGEN RNeasy® mini kit. Samples of cDNA were prepared from total RNA samples and qPCR was carried out on an Applied Biosystems StepOne™ Real-Time PCR System. The formation of double-stranded DNA was monitored by TaqMan® gene expression primers. Expression data were collected as Ct values and the gene expression levels were normalized to the reference control gene, GAPDH.

To investigate the Aggrecan and Collagen type II production of the cells, Alcian blue and immunohistochemistry staining were performed. The chondrogenic pellets were fixed in 10% neutral buffered formalin (VWR, PA, USA), dehydrated through graded alcohol washes (about 70%, about 95% and about 100%) and xylene (EMD, Millipore, USA), embedded in paraffin and sliced into 5 μm thick tissue slices on glass slides. The slides were de-waxed and underwent Hematoxylin and Eosin (H&E) staining for evaluation of cell morphology, Alcian blue staining for detection of proteoglycan production, and immunohistochemistry for Collagen type II detection. For Collagen type II immunohistochemistry, tissue sections were pre-digested with pepsin (1 mg/mL in Tris-HCl, pH 2.0), incubated with the anti-collagen II primary antibody (chemicon, about 1:500) for about 60 minutes, followed by biotinylated goat anti-mouse antibody for 30 minutes, and incubation with streptavidin peroxidase for about 45 minutes at room temperature. Sections were visualized with DAB chromogen, counterstained with Hematoxylin for about 3 minutes, dehydrated, and mounted with Permount solution.

### Results

In order to evaluate the chondrogenic potential of the hiPSC-derived MSC-like cells, chondrogenic differentiation was induced in a 3D, high-density pellet culture (Nejadnik et al., PloS one. 2012; 7(12):e49971). Col2A1, SOX9, and ACAN genes were negative for original hiPSC, mildly positive for hiPSC-derived MSC-like cells and significantly increased in chondrogenic cell pellets at about day 14 of chondrogenic differentiation, which confirms the differentiation of the cells towards chondrogenic lineage (p<0.001) (FIG. 7).

The histologic evaluation of the cell pellets at day 14 of chondrogenic differentiation (about day 42 in FIG. 1) demonstrated cartilage tissue formation on H&E stains (FIGS. 8A and 8B) and positive Alcian blue stains (FIGS. 8C and 8D), indicating proteoglycan production. In addition, collagen type II immunohistochemistry was positive for chondrogenic pellets, indicating production of the hyaline cartilage matrix production of the cells in 3 weeks (FIGS. 8E-8F).

### Example 8

### Engraftment of hiPSC-Derived hMSC and Condrogenic Pellets in Rat Knee Joints

### Materials and Methods

To evaluate in vivo engraftment and exclude in vivo teratoma formations, hiPSC derived MSC-like and chondrocytes were implanted into osteochondral defects of rat knee joints and evaluated with MR imaging and histopathology. The animal experiments were approved by the animal care and use committee at our institution. Osteochondral defects were created in the distal femoral trochlear groove of nine knee joints of five athymic nude Sprague Dawley rats, using a micro-drill (Flash DP Tabletop Micromotor, DBI America Corp, FL, USA). About 2.5×10⁵ hiPSC-derived MSC-like cells (3 knees) or about 2.5×10⁵ hiPSC-derived chondrogenic differentiated cell pellets (3 knees) in about 2 μl of Polyethylene Glycol (PEG) and chondroitin sulfate methacrylate (CS) based scaffold were implanted into the femoral defect. Scaffold only implants (3 knees) served as controls.

The PEG-CS scaffold was prepared freshly every time before the cell implantation by mixing about 14 μl of about 10% PEG3K-DMA solution with about 6 μl of about a 10% CS solution. Directly before implantation, cells in PEG-CS were mixed with about 2.4 μl of polymerizing solution (containing two parts of Ammonium Persulfate (APS) solution (1M) and one part of tetramethylethylenediamine (TMEDA) solution (1M)) and immediately injected to the defect. The cell seeded scaffold polymerized within about 2 minutes.

### Results

In order to exclude teratoma formation and investigate the engraftment of cell implants over time, all knee joints underwent MR imaging directly after stem cell transplantation as well as 3 weeks and 6 weeks after transplantation. MR imaging was performed on a 7 Tesla MR scanner (General Electric “microSigna 7.0”) using a single-channel transmit/receive partial birdcage radiofrequency coil. Sagittal MR images of the rat knees were obtained with a fast spin echo (FSE) sequence (Repetition time, TR: 3000 ms, Echo time, TE: 30 ms) and a multi-echo spin echo (SE) sequence (TR 4000 ms/TE 15, 30, 45, 60 ms), using a field-of-view (FOV) of about 2.5×2.5 cm, a matrix of about 256×256 pixels, and a slice thickness of about 0.5 mm. Since successful engraftment has been characterized by significant decline in T2-relaxation times of cell implants in cartilage defects (Trattnig et al., Investigative Radiology. 2007; 42(6):442-448), pixel-wise T2 relaxation time maps of cell implants were generated using custom research software (Cinetool, GE Global Research Center, Niskayuna, N.Y.). T2 relaxation times of each cell implant was measured on these maps via operator defined regions of interests (ROI). After the last MR scan, animals were sacrificed and specimen were processed for postmortem histopathology correlations, which included H&E stains (FIGS. 11A-11C), immunohistochemistry for collagen type II (FIGS. 11G-11H) and Alcian blue stains (FIGS. 11D-11F).

hiPSC-derived MSC-like cells and hiPSC chondrogenic differentiated cell pellets were implanted into osteochondral defects of the distal femur of nude athymic Sprague Dawley rats. Additional rats with implants of scaffold only served as control group.

The MRI evaluation of the rat knees showed that T2 relaxation times of all implants significantly decreased over time (p<0.001). However, the decline of T2-relaxation times over time and T2 relaxation times at 6 weeks post implantation were significantly higher in cell implants compared to scaffold only (p<0.013) (FIGS. 9A-9I and 10) This signal effect is consistent with successful engraftment as described by Trattnig et al., Investigative Radiology. 2007; 42(6):442-448 and corresponded to H&E (FIGS. 11A-11C) stains showing a higher cellularity and degradation of the scaffold in the cell transplants compared to scaffold only transplants.

H&E staining (FIGS. 11A-11C) confirmed engraftment of hiPSC-derived MSC-like cells and hiPSC chondrogenic differentiated cell pellets in the osteochondral defect. hiPSC-derived MSC-like cell implants had started to remodel the defect and to produce a chondrogenic matrix, as evidenced by positive Alcian blue stains (FIGS. 11D-11F) and positive immunostains for collagen type II (FIGS. 11G-11I). By comparison, scaffold only implants demonstrated no repair of the defect. hiPSC-derived chondrogenic pellets showed stronger GAG and collagen type II staining compared to hiPSC-derived MSC-like cells implants (FIGS. 11D-11I).

We claim:

1. A method for differentiating one or more toti-, pluri-, or multi-potent stem cells into a mesenchymal lineage, the method comprising:
a) culturing the toti-, pluri-, or multi-potent stem cells in a stem cell medium as adherent cells; and
b) pre-differentiating the toti-, pluri-, or multi-potent stem cells into toti-, pluri-, or multi-potent stem cell-derived mesenchymal stem cell (MSC)-like cells in a MSC medium, wherein the method does not result in the formation of embryoid bodies.

2. The method of claim 1, wherein the stem cells are pluripotent stem cells.

3. The method of claim 2, wherein the pluripotent stem cells are induced pluripotent stem cells (iPSCs).

4. The method of claim 3, wherein the iPSCs are generated from one or more human somatic cells.

5. The method of claim 1, wherein the toti-, pluri-, or multi-potent stem cells are cultured in the stem cell medium for about 1 to about 5 days.

6. The method of claim 1, wherein the toti-, pluri-, or multi-potent stem cells are pre-differentiated in the MSC medium for about 3 to about 7 days.

7. The method of claim 6, wherein the toti-, pluri-, or multi-potent stem cells are pre-differentiated in the MSC medium without sub-culturing for about 5 days.

8. The method of claim 1, wherein pre-differentiating the toti-, pluri-, or multi-potent stem cells is initiated when the toti-, pluri-, or multi-potent stem cells reach about 40% to about 60% confluency.

9. The method of claim 1, wherein the toti-, pluri-, or multi-potent stem cells are cultured and pre-differentiated without using a feeder layer.

10. The method of claim 1, wherein the toti-, pluri-, or multi-potent stem cells are cultured and pre-differentiated in a culture vessel having at least one surface coated or treated with a composition that enhances, maintains, or otherwise modifies cells growth, differentiation, and/or attachment of the toti-, pluri-, or multi-potent stem cells.

11. The method of claim 1, wherein the MSC medium comprises a high glucose Dulbecco's Modified Eagle Medium (DMEM) and a fetal bovine serum.

12. The method of claim 1, further comprising the step of differentiating the toti-, pluri-, or multi-potent stem cell-derived MSC-like cells into one or more cells of a mesenchymal lineage.

13. The method of claim 12, wherein the toti-, pluri-, or multi-potent stem cell-derived MSC-like cells are expanded for 1 to 5 passages before being differentiated into one or more cells of a mesenchymal lineage.

14. The method of claim 12, wherein differentiation of the toti-, pluri-, or multi-potent stem cell-derived MSC-like cells is initiated by culturing the cells in a differentiation medium.

15. The method of claim 14, wherein the differentiation medium comprises one or more of a high glucose DMEM, penicillin, streptomycin, L-Glutamine, L-ascorbic acid 2-phosphate sequimagnesium, MEM sodium pyrovate, L-proline, dexamethasone, bovine insulin, sodium selenite, linoleic acid, bovine serum albumin, and TGF-β3.

16. The method of claim 12, wherein the mesenchymal lineage is a chondrogenic lineage.

17. A composition for the treating a cartilage disease or disorder comprising:
one or more chondrocytes produced by the method comprising:
a) culturing toti-, pluri-, or multi-potent stem cells in a stem cell medium as adherent cells;
b) pre-differentiating the toti-, pluri-, or multi-potent stem cells into toti-, pluri-, or multi-potent stem cell-derived mesenchymal stem cell (MSC)-like cells in a MSC medium, wherein the method does not result in the formation of embryoid bodies; and
c) differentiating the toti-, pluri-, or multi-potent stem cell-derived MSC-like cells into one or more cells of a mesenchymal lineage.

18. The composition of claim 17, further comprising an active agent selected from the group consisting of immunomodulators, analgesics, anesthetics, anti-inflammatory agents, antibodies, aptamers, RNA, DNA, anti-infective agents, blood derivatives, blood formation agents, coagulation agents, and diagnostic agents.

19. The composition of claim 17, further comprising a matrix.

20. The composition of claim 17, further comprising a hydrogel.

21. The composition of claim 17, further comprising a pharmaceutically acceptable carrier.

22. A method for treating a cartilage disease or disorder, comprising: delivering a composition to a joint affected by the cartilage disease or disorder, wherein the composition comprises one or more chondrocytes produced by the method comprising:
a) culturing the toti-, pluri-, or multi-potent stem cells in stem cell media as adherent cells;
b) pre-differentiating the toti-, pluri-, or multi-potent stem cells into toti-, pluri-, or multi-potent stem cell-mesenchymal stem cell (MSC)-like cells in MSC medium, wherein the method does not result in the formation of embryoid bodies; and
c) differentiating the toti-, pluri-, or multi-potent stem cell-derived MSC-like cells into one or more cells of a mesenchymal lineage.

23. The method of claim 22, wherein the disease or disorder is osteoarthitis.

24. The method of claim 22, wherein the composition is delivered to the joint via intra-articular injection.

25. The method of claim 22, wherein the compositions is delivered to the joint during a surgery.
